(19) United States
(12) Patent Application Publication
(10) Pub. No.: US 2024/0318214 A1
(43) Pub. Date: Sep. 26, 2024

(54) A GENETICALLY ENGINEERED BACTERIUM AND A PREPARATION METHOD AND USE THEREOF

(71) Applicant: SYNAURA BIOTECHNOLOGY (SHANGHAI) CO., LTD., Shanghai (CN)

(72) Inventors: Yan WU, Shanghai (CN); Jing TANG, Shanghai (CN); Jin ZHAO, Shanghai (CN); Shu WANG, Shanghai (CN); Zhenhua TIAN, Shanghai (CN); Fei YAO, Shanghai (CN); Miao LI, Shanghai (CN); Hong XU, Shanghai (CN); Chenxi HUANG, Shanghai (CN); Yurou LIU, Shanghai (CN)

(73) Assignee: SYNAURA BIOTECHNOLOGY (SHANGHAI) CO., LTD., Shanghai (CN)

(21) Appl. No.: 18/576,607

(22) PCT Filed: Oct. 12, 2022
(86) PCT No.: PCT/CN2022/124826

§ 371 Date: Jan. 4, 2024

(30) Foreign Application Priority Data
Dec. 10, 2021 (CN) 202111509981.9

(51) Int. Cl. C12P 19/04; C12N 1/20; C12N 9/10; C12N 15/70; C12P 19/18; C12R 1/19

(52) U.S. Cl. CPC C12P 19/04; C12N 1/20; C12N 9/1051; C12N 15/70; C12P 19/18; C07K 2319/24; C07K 2319/35; C12R 2001/19; C12Y 204/01069

(57) ABSTRACT

A genetically engineered bacterium and a preparation method and use thereof are disclosed. The genetically engineered bacteria contain a gene encoding α-1,2-fucosyltransferase, and a gene encoding a protein tag is connected to the gene encoding α-1,2-fucosyltransferase; the protein tag is MBP, SUMO1, SUMO2 or TrxA, the amino acid sequence of the MBP is shown in SEQ ID NO: 2, the amino acid sequence of the SUMO1 is shown in SEQ ID NO: 3, the amino acid sequence of the SUMO2 is shown in SEQ ID NO: 4, the amino acid sequence of the TrxA is shown in SEQ ID NO: 5. Fermentation with the genetically engineered bacteria can greatly increase the yield of 2′-fucosyllactose compared to the genetically engineered bacteria that only expresses α-1,2-fucosyltransferase exogenously, and the yield can be more than doubled in a preferred case.

### CROSS-REFERENCE TO RELATED APPLICATION

This application is a U.S. National Phase Entry of International Application No. PCT/CN2022/124826 filed on Oct. 12, 2022, which claims priority to Chinese Patent Application No. 202111509981.9 filed on Dec. 10, 2021, which are hereby incorporated herein by reference in their entireties.

### TECHNICAL FIELD

The invention relates to the field of microbial engineering, and in particular relates to a genetically engineered bacterium and a preparation method and use thereof.

### BACKGROUND

Human milk oligosaccharide (HMO) is one of the components with high nutritional value in human milk. According to the monosaccharide composition and structural characteristics, HMOs can be categorized into neutral fucosyl, neutral non-fucosyl, sialic acid, etc. Among them, 2′-fucosyllactose (2′-fucosyllactose, 2′-FL) is the oligosaccharide with the highest content in human milk, and it is also one of the first HMOs approved by FDA and EU to be added to infant milk powder, dietary supplements and medical foods. 2′-FL has various functional activities such as regulating intestinal microbiome, preventing the adhesion of pathogenic bacteria, immunomodulating, and promoting the development and repair of the nervous system.

The main synthesis methods of 2′-FL include chemical synthesis, whole-cell synthesis and enzymatic synthesis, but there are many difficulties in the actual production process of chemical synthesis or enzymatic synthesis, such as stereochemical control, specific linkage formation, availability of raw materials, etc., synthesis with biosynthetic technology through microbial metabolism is more economical and efficient compared with chemical synthesis and enzymatic synthesis. GDP-fucose is synthesized from carbon sources such as glucose or glycerol using biosynthetic methods to simulate the metabolic mechanism of microorganisms themselves (or simulation), meanwhile fucosyl is transferred to lactose by exogenously expressed α-1,2-fucosyltransferase. This is the main method for industrial production of 2′-FL.

Since the lack of an appropriate post-translational processing mechanism in prokaryotic expression system, in the process of expressing exogenous proteins in Escherichia coli as the host bacteria, insoluble inclusion bodies will be formed due to incorrect protein folding, which again requires complex denaturation and renaturation, making it difficult to express large amounts of soluble exogenous proteins.

The fusion protein tag refers to the fusion of a protein sequence at the N-terminus or C-terminus of the protein, the purpose of which is to enhance the soluble expression of the recombinant protein, so as to improve the expression level of the recombinant protein in E. coli. Fusion protein tags provide an efficient strategy for the soluble expression of exogenous proteins in E. coli, but as there are many factors that result in the non-expression or very low levels of expression of exogenous protein in E. coli, such as the formation of inactive inclusion bodies due to incorrect folding during translation, or the formation of incorrectly paired disulfide bonds resulting in unstable protein expression, there may be different effects for different protein tags on promoting the expression of exogenous proteins in E. coli.

Patent CN112322565A of Jiangnan University discloses a method for improving the yield of 2′-fucosyllactose in recombinant Escherichia coli, which uses flexible linker to tag four different proteins: maltose binding protein (MBP), thioredoxin A (TrxA), ubiquitin-related small modification protein (SUMO), and transcription termination anti-termination factor (NusA), respectively fused to the N-terminus of α-1,2-fucosyltransferase FutC, and the constructed fusion protein FP-futC can increase the yield of 2′-FL from the catalyzed synthesis through to different levels. Among them, the yield of 2′-FL synthesized by TrxA-futC fusion protein was the highest, reaching 2.94 g/L, and the yield of 2′-FL synthesized by SUMO-futC fusion protein was 2.56 g/L. The TrxA-futC fusion protein gene was further integrated into the yjiP site on the genome of Escherichia coli MG1655 to obtain a plasmid-free 2′-FL genetically engineered strain MG-26ΔyjiP::trxA-futC, and the yield of 2′-FL after shake flask fermentation reached 3.85 g/L.

But the efficiency of producing 2′-fucosyllactose by the genetically engineered bacteria in the prior art is still not high enough, especially the yield is low during de novo synthesis.

### SUMMARY

In view of the technical defects in the prior art, such as the low efficiency of the preparation method of 2′-fucosyllactose (2′-FL) and the poor function of the genetically engineered bacteria for producing 2′-fucosyllactose, the present invention provides a genetically engineered bacterium and a preparation method of 2′-fucosyllactose. The genetically engineered bacteria modulate the expression of some genes in the starting bacteria (such as Escherichia coli), especially by adding a protein tag to increase the expression of α-1,2-fucosyltransferase, so as to obtain a high-yield genetically engineered bacterium for 2′-fucosyllactose.

In order to solve the above-mentioned technical problems, a technical solution provided by the present invention is: a genetically engineered bacterium containing a gene encoding α-1,2-fucosyltransferase, and a gene encoding a protein tag is connected to the gene encoding α-1,2-fucosyltransferase (α-1,2-fucosyltranferase, abbreviated as futC in the present invention); the protein tag is MBP, SUMO1, SUMO2 or TrxA, the amino acid sequence of MBP is shown in SEQ ID NO: 2, the amino acid sequence of SUMO1 is shown in SEQ ID NO: 3, the amino acid sequence of SUMO2 is shown in SEQ ID NO: 4, the amino acid sequence of TrxA is shown in SEQ ID NO: 5.

In a preferred embodiment of the present invention, the amino acid sequence of the α-1,2-fucosyltransferase is shown in SEQ ID NO: 1.

In a specific embodiment of the present invention, the nucleotide sequence of the gene encoding the α-1,2-fucosyltransferase is shown in SEQ ID NO: 6.

In a preferred embodiment of the present invention, the nucleotide sequence of the gene encoding the MBP is shown in SEQ ID NO: 7, and the nucleotide sequence of the gene encoding the SUMO1 is shown in SEQ ID NO: 8, the nucleotide sequence of the gene encoding the SUMO2 is shown in SEQ ID NO: 9, and the nucleotide sequence of the gene encoding the TrxA is shown in SEQ ID NO: 10.

In a preferred embodiment of the present invention, the GDP-fucose degradation pathway of the genetically engineered bacteria is blocked. Preferably, all or part of the genes in the GDP-fucose degradation pathway in the genetically engineered bacteria are knocked out. More preferably, the wcaJ gene of the genetically engineered bacteria is knocked out.

In a preferred embodiment of the present invention, the GDP-mannose degradation pathway of the genetically engineered bacteria is blocked. Preferably, all or part of the genes in the GDP-mannose degradation pathway of the genetically engineered bacteria are knocked out. More preferably, the nudD and/or nudK genes of the genetically engineered bacteria are knocked out.

In a preferred embodiment of the present invention, the gene LacZ encoding the lactose operon beta-galactosidase of the genetically engineered bacteria is knocked out.

In a preferred embodiment of the present invention, the protein tag is located at the N-terminus of the α-1,2-fucosyltransferase.

In a specific embodiment of the present invention, the gene encoding the protein tag and the α-1,2-fucosyltransferase gene are linked together on a plasmid vector. Preferably, the plasmid is pET28a.

In a specific embodiment of the present invention, the starting bacteria of the genetically engineered bacteria is Escherichia coli, preferably BL21 strain.

In a preferred embodiment of the present invention, the genetically engineered bacteria overexpress one or more of the manC, manB, gmd and wcaG genes, and the amino acid sequences encoded by the manC, manB, gmd and wcaG genes are respectively shown in SEQ ID NOs: 95-98. Preferably, the nucleotide sequences of the manC, manB, gmd and wcaG genes are respectively shown in SEQ ID NOs: 91-94.

In the present invention, the manC gene is a mannose-1-phosphate guanylyltransferase gene. The manB gene is a phosphomannose mutase gene. The gmd gene is a GDP-D-mannose-4,6-dehydratase gene. The wcaG is a GDP-4-keto-6-deoxy-D-mannose-3,5-epimerase-4-reductase gene.

In order to solve the above-mentioned technical problems, a technical solution provided by the present invention is: a preparation method of 2′-fucosyllactose, which comprises: using lactose as a substrate, glycerol or glucose as a carbon source, fermenting the genetically engineered bacteria as described in the present invention, obtaining the 2′-fucosyllactose; preferably, the fermentation medium is TB medium.

In a preferred embodiment of the present invention, when the genetically engineered bacteria are fermented to an OD600 of 0.6-0.8, IPTG with a final concentration of 0.1-0.5 mM is added to the reaction system.

In a preferred embodiment of the present invention, the concentration of the glycerol or glucose is 5-50 g/L of glycerol, and the concentration of the lactose is 5-20 g/L.

In a specific embodiment of the present invention, when IPTG is added, the temperature of the fermentation is adjusted to 20-30° C., and the stirring is performed at a rotation speed of 150-300 rpm.

In a preferred embodiment of the present invention, a step of preparing the seed solution is further incorporated before the catalysis. Preferably, the step of preparing the seed solution comprises culturing the genetically engineered bacteria in LB medium. More preferably, the volume ratio of the seed liquid used in the fermentation to the liquid is 1:100.

In order to solve the above-mentioned technical problems, a technical solution provided by the present invention is: a recombinant expression vector, which comprises a gene encoding a protein tag and a gene encoding α-1,2-fucosyltransferase, and the protein tag is MBP, SUMO1, SUMO2 or TrxA, the amino acid sequence of the MBP is shown in SEQ ID NO: 2, the amino acid sequence of the SUMO1 is shown in SEQ ID NO: 3, and the amino acid sequence of SUMO2 is shown in SEQ ID NO: 4, the amino acid sequence of the TrxA is shown in SEQ ID NO: 5.

In a preferred embodiment of the present invention, the amino acid sequence of the α-1,2-fucosyltransferase is shown in SEQ ID NO: 1.

In a specific embodiment of the present invention, the nucleotide sequence of the gene encoding the MBP is shown in SEQ ID NO: 7, and the nucleotide sequence of the gene encoding the SUMO1 is shown in SEQ ID NO: 8, and the nucleotide sequence of the gene encoding the SUMO2 is shown in SEQ ID NO: 9, and the nucleotide sequence of the gene encoding the TrxA is shown in SEQ ID NO: 10.

In a specific embodiment of the present invention, the nucleotide sequence of the gene encoding the α-1,2-fucosyltransferase is shown in SEQ ID NO: 6;

In a specific embodiment of the present invention, the starting vector of the recombinant expression vector is pET28a plasmid vector.

In order to solve the above-mentioned technical problems, a technical solution provided by the present invention is: a method for preparing the genetically engineered bacteria of the present invention, comprising: transferring the recombinant expression vector of the present invention into Escherichia coli to obtain the genetically engineered bacteria.

In a preferred embodiment of the present invention, the method further comprises: knocking out the LacZ, wcaJ, nudD and/or nudK genes in the E. coli.

In a preferred embodiment of the present invention, the method further comprises: making the E. coli to overexpress manC, manB, gmd and/or wcaG genes, the amino acid sequences encoded by the manC, manB, gmd and wcaG genes are respectively shown in SEQ ID NOs: 95-98.

In a specific embodiment of the present invention, the Escherichia coli is a BL21 strain.

In a preferred embodiment of the present invention, the method further comprises: knocking out the LacZ, wcaJ, nudD and/or nudK genes in the E. coli.

In a preferred embodiment of the present invention, the method further comprises: making the E. coli to overexpress manC, manB, gmd and/or wcaG genes, the amino acid sequences encoded by the manC, manB, gmd and wcaG genes are respectively shown in SEQ ID NOs: 95-98.

In order to solve the above-mentioned technical problems, a technical solution provided by the present invention is: the use of the genetically engineered bacteria as described in the present invention or the recombinant expression vector as described in the present invention in the preparation of fucosyllactose, the fucosyllactose is preferably 2′-fucosyllactose.

On the basis of conforming to common knowledge in the art, the above preferred conditions can be combined arbitrarily to obtain preferred examples of the present invention.

The reagents and raw materials used in the present invention are all commercially available.

The positive progressive effect of the present invention lies in:

When the genetically engineered bacteria described in the present invention expresses the preferred α-1,2-fucosyltransferase of the present invention linked with a protein tag, it can greatly increase the 2′-fucosyllactose compared with the genetically engineered bacteria that only express α-1,2-fucosyltransferase exogenously, and the yield can be more than doubled in a preferred case.

### BRIEF DESCRIPTION OF THE DRAWINGS

FIG. 1 is a profile of the lacZ knockout verification;

FIG. 2 is a profile of pTargetF plasmid;

FIG. 3 is a profile of RSF-CBDG plasmid;

FIG. 4 is a graph showing the detection of 2′-FL content in FLIS202 fermentation broth.

### DETAILED DESCRIPTION

In order to further illustrate the technical means adopted by the present invention and effects thereof, the following detailed description is given in conjunction with the accompanying drawings and the preferred examples of the present invention. The experimental methods in the following examples with no specific conditions are selected according to conventional methods and conditions, or according to the product insert.

BL21 (DE3) strain was purchased from Novagen Company, Cat. #69450-M; Escherichia coli Trans 10 competent cells were purchased from Beijing TransGen Biotech Co., Ltd.; plasmid extraction kit and gel recovery kit were purchased from Sangon Biotech (Shanghai) Co., Ltd., and SDS-PAGE kit was purchased from Shanghai Epizyme Biomedical Technology Co., Ltd.

In the examples, a high performance liquid chromatography (HPLC) system (SHIMADZU LC-20AD XR) was used to quantitatively detect the synthesis of 2′-FL in the fermentation broth of recombinant Escherichia coli, and the concentrations of 2′-FL and the substrate lactose in the fermentation broth were determined by HP-Amide column (Sepax, 4.6×250 mm 5 μm). The HPLC detector was a differential detector, the detection temperature of the chromatographic column was set to 35° C., the mobile phase was eluted by acetonitrile:water=68:32, and the detection flow rate was 1.4 ml/min.

### Example 1 Construction of Chassis Strain FLIS009

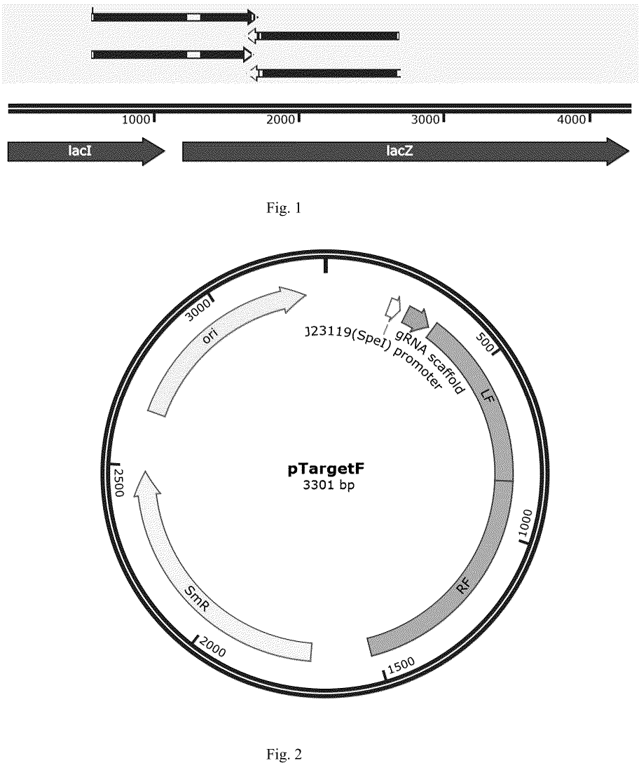

1.1 Construction of Small Guide RNA (sgRNA) Plasmid for CRISPR/Cas9 Knockout System
(1) The primers designed according to Table 3 (synthesized by Tsingke) were used for the specific amplification of each fragment using the pTargetF plasmid (see FIG. 2 for the profile) or the BL21 genome as a template, and the high-fidelity enzyme Primer Star Mix of Takala Company was used for PCR reaction, the reaction system is shown in the following Table 1:

TABLE 1
PCR amplification reaction system

| | Volume added to the | |
|---|---|---|
| Agent | PCR reaction system | |
| cDNA | 1 | μl |
| Primer F | 1 | μl |
| Primer R | 1 | μl |
| PCR Mix | 12.5 | μl |
| ddH₂O | 9.5 | μl |

The PCR amplification procedure is shown in the following Table 2:

TABLE 2
PCR reaction procedure

| Temperature | Time | | Cycles |
|---|---|---|---|
| 98° C. | Pre-denaturation | | |
| | 3 min | | |
| 98° C. | Denaturation | | |
| | 15 s | | |
| 55° C. | Annealing 15 | | |
| | | {close oversize brace} | 34 cycles |
| 72° C. | Extension | | |
| | 0.1 kb/s | | |
| 72° C. | Extension | | |
| | 5 min | | |
| 12° C. | Insulation | | |

5 μl of the amplified product was subjected to 1% agarose electrophoresis to detect the amplification result. The target fragments were recovered by cutting gel using a gel recovery kit. The target fragments were ligated and recombined using NEB's multi-fragment recombinase, and the ligated recombination products were transformed into E. coli competent cells Trans 10. Sterilized LB liquid medium was added, cultured at 37° C. with shaking at 250 rpm for 1 h;
(2) The spot was picked onto the LB solid plate with spectinomycin added in advance, and inverted overnight at 37° C.;
(3) After the white single colony has grown, the white single colony was picked into a centrifuge tube containing 2 ml of LB liquid medium (containing 50 μg/ml spectinomycin), and cultured at 37° C. with shaking at 180 rpm for 6 hours;
(4) PCR detection was carried out on the bacterial liquid, 500 μl of the bacterial liquid verified as positive was sent to Tsingke Company for sequencing, and the remaining bacterial liquid was stored in 20% glycerol.
(5) The strains that were verified through sequencing were subjected to expanded culturing, and plasmid extraction was carried out by a plasmid extraction kit from Sangon. The sgRNA plasmids containing the BL21 genome were obtained and named as pTargetF-ΔLacZ, pTargetF-ΔnudK, pTargetF-ΔnudD, pTargetF-ΔwcaJ, respectively.

TABLE 3
Primer information for lacZ, nudK, nudD, wcaJ, etc gene knockout sgRNA
plasmid construction

| | | | | | | Product |
|---|---|---|---|---|---|---|
| | | Primer | | SEQ ID | | Size |
| Plasmid | Gene | Name | Sequence | NO: | Template | (bp) |
| pTarget | pTarget | GA001- | tgccgaccgtctagagtcgacctgca | 11 | pTargetF | 2000 bp |
| F- | F | P1-F4 | gaagcttag | | | |
| ΔLacZ | Backbone | GA001- | aacTGGCGTTACCCAACT | 12 | | |
| | 1 | P1-R4 | TAATCactagtattatacctaggac | | | |
| | | | tg | | | |
| | PAM1 | GA001- | tagtGATTAAGTTGGGTA | 13 | pTargetF | 150 bp |
| | | P1-F1 | ACGCCAgttttagagctagaaata | | | |
| | | | gcaag | | | |
| | | GA001- | gttccggaattcaaaaaaagcaccga | 14 | | |
| | | P1-R1 | ctcggtgcc | | | |
| | LF | GA001- | gctttttttgaattccggaacgggaagg | 15 | BL21 | 560 bp |
| | | LF | cgactggagtg | | Genome | |
| | | GA001- | ggtgcgggcctcgacggccagtgaat | 16 | | |
| | | P1-LR | ccgtaatcatg | | | |
| | RF | GA001- | tcgactctagacggtcggcaaagacc | 17 | BL21 | 1800 bp |
| | | P1-RR | agaccgttc | | Genome | |
| | | GA001- | ctggccgtcgaggcccgcaccgatc | 18 | | |
| | | P1-RF | gcccttc | | | |
| pTarget | Donor- | nudK- | tgaattcttcccttcctgaatcatctgca | 19 | BL21 | 500 bp |
| F- | LF | f2 | aaaac | | Genome | |
| ΔnudK | | nudK- | gtggagtcggtaaaataacaataatatt | 20 | | |
| | | R2 | tcgttg | | | |
| | Donor- | nudK- | attgttattttaccgactccacagcgcg | 21 | BL21 | 500 bp |
| | RF | F3 | aaatgaac | | Genome | |
| | | nudK- | ctagaccggaagagccgtttatcaata | 22 | | |
| | | R3 | cc | | | |
| | pTarget | nudK- | gataaacggctcttccggtctagagtc | 23 | pTargetF | 2000 bp |
| | F | F4 | gacctgcagaag | | | |
| | Backbone | nudK- | ctaaaacGCGCAGCTTTCA | 24 | | |
| | | R4 | ATCAGCTGactagtattatacct | | | |
| | | | aggactgag | | | |
| | PAM | nudK- | ctagtCAGCTGATTGAAA | 25 | pTargetF | 150 bp |
| | | F1 | GCTGCGCgttttagagctagaaa | | | |
| | | | tagcaagttaa | | | |
| | | nudK- | gattcaggaagggaagaattcaaaaa | 26 | | |
| | | R1 | aagcaccgactcggtgccac | | | |
| pTarget | Donor- | pT- | gctttttttgaattccacttcgtaatcctg | 27 | BL21 | 500 bp |
| F- | LF | nudD- | aatatgcag | | Genome | |
| ΔnudD | | F2 | | | | |
| | | PT- | cgctccactgattaccactggctgacg | 28 | | |
| | | nudD- | ccggacgcac | | | |
| | | R2 | | | | |
| | Donor- | PT- | ccagtggtaatcagtggagcgcacta | 29 | BL21 | 500 bp |
| | RF | nudD- | ccgtggcaaagtcttcc | | Genome | |
| | | F3 | | | | |
| | | pT- | cgactctagagacaacttccacccga | 30 | | |
| | | nudD- | gtaattcgcatgtg | | | |
| | | R3 | | | | |
| | PAM1 | pT- | ctagtgtgagtggtgaaatccgtgcgtt | 31 | pTargetF | 150 bp |
| | | nudD- | ttagagctagaaatagcaag | | | |
| | | F1 | | | | |
| | | pT | gattacgaagtggaattcaaaaaaagc | 32 | | |
| | | nudD- | accgactcgg | | | |
| | | R1 | | | | |
| | pTarget | pT- | ggtggaagttgtctctagagtcgacct | 33 | pTargetF | 2000 bp |
| | F | nudD- | gcagaagcttag | | | |
| | Backbone | F4 | | | | |
| | | PT- | ctaaaacgcacggatttcaccactcac | 34 | | |
| | | nudD- | actagtattatacctaggactgagc | | | |
| | | R4 | | | | |
| pTarget | Donor- | wcaJ-F2 | cgagtcggtgctttttttgaattcgacag | 35 | BL21 | 550 bp |
| F- | LF | | cggcatgatcccgtggctg | | Genome | |
| ΔwcaJ | | wcaJ-R2 | cgccacgccagcccaacaggtgcat | 36 | | |
| | | | gtagaggaatg | | | |
| | Donor- | wcaJ-F3 | catgcacctgttgggctggcgtggcg | 37 | BL21 | 550 bp |
| | RF | | aaaccgacacg | | Genome | |
| | | wcaJ-R3 | cagggtaatagatctaagcttgcgcgg | 38 | | |
| | | | aactgctgtccgtgggg | | | |
| | PAM1 | wcaJ-F1 | gtcctaggtataatactagtcatcgccg | 39 | pTargetF | 150 bp |
| | | | cagcggtttcaggttttagaget | | | |
| | | wcaJ-R1 | cagccacgggatcatgccgctgtcga | 40 | | |
| | | | attcaaaaaaagcaccgactcg | | | |
| | pTarget | wcaJ-F4 | ccccacggacagcagttccgcgcaa | 41 | pTargetF | 2000 bp |
| | F | | gcttagatctattaccctg | | | |
| | Backbone | wcaJ-R4 | gctctaaaacctgaaaccgctgcggc | 42 | | |
| | | | gatgactagtattatacct | | | |

(1) Preparation of BL21 competent cells: single colony streak culture was performed on the strain BL21 stored at −80° C.; a single colony was picked and inoculated to 5 ml of LB medium, and cultured at 37° C. with shaking at 200 rpm until the OD was about 0.5 (about 3 h), then the culture was ice-bathed for 30 min; the bacterial liquid was transferred to a pre-cooled sterile centrifuge tube, centrifuged at 4000 rpm for 10 min at 4° C., the supernatant was discarded, and the bacteria was collected; the cells were resuspended with pre-cooled sterile water, centrifuged at 4000 rpm for 10 min at 4° C., the supernatant was discarded; the cells were resuspended twice with a solution containing 0.1 M CaCl₂), centrifuged at 4000 rpm for 10 min at 4° C., and the supernatant was discarded; finally, the cells were resuspended with an appropriate amount of 0.1 M CaCl₂) solution containing 15% glycerol, dispensed into 1.5 ml centrifuge tubes with 100 ul per tube, quickly frozen in liquid nitrogen, and stored at −80° C.
(2) 3 ul pCas-sac plasmid was added to 100 μL E. coli BL21 competent, placed on ice for 30 min, then heat-shocked at 42° C. for 45 s, and immediately placed on ice for 2-5 min; after adding 800 μL of LB, it was placed on a shaker at 30° C. and incubated for 45 min, followed by plating (Km resistant, LB medium), and was placed upside down in a 30° C. incubator, and cultured overnight; spots were picked to LB medium (Kana resistant), cultured for several hours before bacteria preservation (final concentration of glycerol 30%).
(3) The pCas-sac/BL21 transformants were picked and inoculated into LB sieve tubes (Kana resistant) and cultured at 30° C. until OD=0.2, then arabinose with a final concentration of 2 g/L was added for induction, and at OD=0.4, the competent preparation was carried out, the preparation method is the same as operation (1);
(4) The correctly constructed pTargetF-ΔLacZ plasmids were transformed into pCas-sac/BL21 competent cells by heat shock method, coated on LB plates (k+, spe+) after recovery, and cultured at 30° C. overnight;
(5) PCR verification was carried out on a single colony on the resistant plate, with verification primers shown in Table 4, and the sequencing verification profile shown in FIG. 1, and the LacZ gene knockout strain was verified;
(6) The strains with LacZ gene knockout were picked and shaken, and rhamnose with a final concentration of 10 mM was added to induce the loss of the sgRNA plasmid pTargetF-ΔLacZ;
(7) Streaking to verify whether the pTargetF-ΔLacZ plasmid was lost (see Table 4 for primers), and the LacZ gene knockout strains with sgRNA loss were named as FLIS001.
   1.2.2 Knockout of GDP-Fucose Degradation Related Gene wcaJ Based on FLIS001 Strain

FLIS001 competent preparation and knockout were the same as in 1.2.1. The pTargetF-ΔwcaJ plasmid was used to knock out the wcaJ gene. The method was the same as that in 1.2.1, the wcaJ gene knockout strain was obtained and named as FLIS007.

1.2.3 Knockout of GDP-Mannose Degradation Related Genes nudD and nudK Based on FLIS007 Strain
(1) The nudD gene in the FLIS007 strain was knocked out using the pTargetF-ΔnudD plasmid, and the method is the same as that in (1), the knockout strain was named as FLIS008.
(2) The nudK gene was knocked out on the basis of the FLIS008 strain using the pTargetF-ΔnudK plasmid, and the method is the same as that in 1.2.1, the knockout strain was named as as FLIS009.
(3) Loss of sgRNA plasmid was performed in FLIS009 strain, the method is the same as that in 1.2.1.
(4) Loss of pCas-SAC plasmid was performed in FLIS009 strain: the FLIS009 strain with sgRNA loss was inoculated on an antibiotic free LB plate containing 10 g/L sucrose, cultured at 37° C., and PCR validation was performed with pCas-SAC verification primers in Table 4 to ensure that the pCas-SAC plasmid free chassis strain FLIS009 was obtained.

TABLE 4
Gene knockout validation primers for LacZ, wcaJ, nudD, nudK and the like

| | | | | SEQ | Product |
|---|---|---|---|---|---|
| | Knockout | Primer | | ID | Size |
| Strain | Gene | Name | Primer Sequence | NO: | (bp) |
| FLIS001 | LacZ | LacZ-YZ1- | cgcgctgttagcgggcccattaagttctg | 43 | 2000 bp after |
| | | F | | | knockout |
| | | LacZ-YZ2- | ggtcttcatccacgcgcgcgtacatcgg | 44 | |
| | | R | | | |
| FLIS007 | wcaJ | wcaJ-YZ- | gtcggcctgttggcagaagcattc | 45 | 1.5 kb after |
| | | for | | | knockout |
| | | wcaJ-YZ- | gtagccaaacagcagcgttcttaccgcac | 46 | |
| | | R | | | |
| FLIS008 | nudD | nudD-YZ-F | ccgtcgccagctgtgccactttg | 47 | 1.2 kb after |
| | | nudD-YZ-R | caaactgtgcgaatcttacaatcgcc | 48 | knockout |
| FLIS009 | nudK | nudK-YZ-F | gctgagcatcaataaacaacaacgctg | 49 | 1.4 kb after |
| | | nudK-YZ-R | atgaagatgcgccgggcgtttatg | 50 | knockout |
| sgRNA | | CX-targetF- | cagcgagtcagtgagcgag | 51 | 2000 bp |
| Plasmid | | F | | | |
| | | CX-targetF- | gacattgcactccaccgct | 52 | |
| | | R | | | |
| pCas- | | Kan-F | gaaggagaaaactcaccgag | 53 | 3300 bp |
| SAC | | Pcr4-R1 | cagctgcataaaattgcgattggcaaaacc | 54 | |
| | | | atc | | |

### Example 2 Production of 2′-FL Using FLIS009 Strain

### 2.1 Construction of Expression Plasmid for 2′-FL Synthesis

2.1.1 Construction of Plasmid pRSF-CBDG

manC gene is a mannose-1-phosphate guanylyltransferase gene; manB gene is a phosphomannose mutase gene; gmd gene is a GDP-D-mannose-4,6-dehydratase gene; wcaG is a GDP-4-keto-6-deoxy-D-mannose-3,5-epimerase-4-reductase gene.

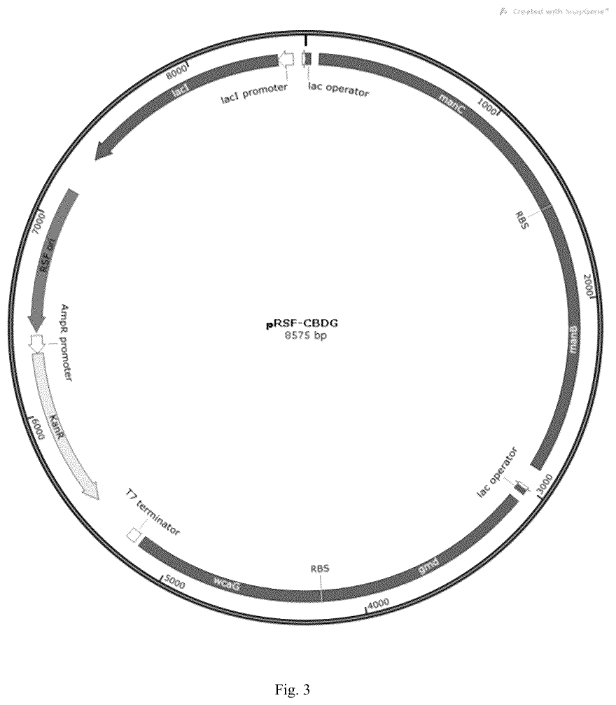

The primers designed according to Table 5 (synthesized by Tsingke) were used for the specific amplification of each fragment using the pRSFDuet plasmid or the BL21 genome as the template. See 1.1 for the amplification method.
(2) The recovery, ligation and recombination, competent transformation and ampicillin resistance screening of amplified products were carried out according to the method in 1.1;
(3) Positive colonies were selected for PCR verification, 500 μl of the bacterial liquid verified as positive was sent to Tsingke Company for sequencing, and the remaining bacterial solution was stored in 20% glycerol.
(4) The strains that were verified through sequencing were subjected to expanded culturing, and plasmid extraction was carried out by a plasmid extraction kit from Sangon to obtain a plasmid containing manC, manB, gmd, and wcaG genes, which is named as pRSF-CBDG plasmid (see FIG. 3).

TABLE 5
Primer information for plasmid RSF-CBDG construction

| | | | | SEQ | | Product |
|---|---|---|---|---|---|---|
| | | Primer | | ID | | Size |
| Plasmid | Gene | Name | Primer Sequence | NO: | Template | (bp) |
| pRSF- | manC | CBGW- | taaggagatataccatggcgcagtcgaa | 55 | BL21 | 1437 bp |
| CBDG | | manC-F | actcta | | Genome | |
| | | CBGW- | gttaattttttcatggtatatctccttttacacc | 56 | | |
| | | manC-R | cgtccgtagcg | | | |
| | manB | CBGW- | cggacgggtgtaaaaggagatataccat | 57 | BL21 | 1371 bp |
| | | manB-F | gaaaaaattaacctgctttaaag | | Genome | |
| | | CBGW- | gagctcgaattcttactcgttcagcaacgt | 58 | | |
| | | manB-R | cag | | | |
| | gmd | CBGW- | gaaggagatatacaatgtcaaaagtcgct | 59 | BL21 | 1122 bp |
| | | gmd-F | ctcat | | Genome | |
| | | CBGW- | gttgtttactcatggtatatctccttttatgac | 60 | | |
| | | gmd-R | tccagcgcgatcg | | | |
| | wcaG | CBGW- | ctggagtcataaaaggagatataccatga | 61 | BL21 | 966 bp |
| | | wcaG-F | gtaaacaacgagtttttattg | | Genome | |
| | | CBGW- | tggcagcagcctaggttacccccgaaag | 62 | | |
| | | wcaG-R | cggtct | | | |
| | pRSF | CBGW- | gctttcgggggtaacctaggctgctgcca | 63 | pRSFDuet | 3395 bp |
| | Duet | F1 | ccg | | | |
| | Backbone | CBGW- | cgactgcgccatggtatatctccttattaaa | 64 | | |
| | 1 | R1 | gttaaac | | | |
| | pRSF | CBGW- | ttgctgaacgagtaagaattcgagctcgg | 65 | pRSFDuet | 187 bp |
| | Duet | F2 | cgcg | | | |
| | Backbone | CBGW- | gcgacttttgacattgtatatctccttcttata | 66 | | |
| | 2 | R2 | cttaac | | | |

The amino acid sequences of manC, manB, gmd and wcaG are respectively shown in SEQ ID NOs: 95-98, and the nucleotide sequences are respectively shown in SEQ ID NOs: 91-94.

2.1.2 Construction of α-1,2-Fucosyltransferase futC Expression Plasmid

α-1,2-fucosyltransferase futC (GT007), MBP, SUMO1, SUMO2, TrxA sequences (amino acid sequences are respectively shown in SEQ ID NOs: 1-5, nucleotide sequences are respectively shown in SEQ ID NOs: 6-10) were synthesized by Sangon Company. The primers designed according to Table 6 (synthesized by Tsingke) were used for the specific amplification of each fragment using the pET28a plasmid or the BL21 genome as the template. See 1.1 for the amplification method.
(1) The recovery of amplified products, ligation and recombination, competent transformation and Kana resistance screening were carried out according to the method in 1.1;
(2) Positive colonies were selected for PCR verification, 500 μl of the bacterial liquid that was verified to be positive was sent to Tsingke Company for sequencing, and the remaining bacterial solution was stored in 20% glycerol.
(3) The strains that were verified through sequencing were subjected to expanded culturing, and plasmid extraction was carried out by a plasmid extraction kit from Sangon to obtain futC expression plasmids with different tags, which are named as pET-MBP-futC, pET-SUMO1-futC, pET-SUMO2-futC, pET-TrxA-futC plasmid, pET-futC, respectively.

TABLE 6
Primer information for futC expression plasmid construction

| | | | | SEQ | | Product |
|---|---|---|---|---|---|---|
| | | Primer | | ID | | Size |
| Plasmid | Gene | Name | Primer Sequence | NO: | Template | (bp) |
| pET- | MBP | FL121- | AAGGAGATATACCATGaaaatc | 67 | MBP | 1203 bp |
| MBP- | | MBP-F | gaagaaggtaa | | | |
| futC | | FL121- | GATGCTCATatGGAATTcggatc | 68 | | |
| | | MBP-R | cctgaaaat | | | |
| | futC | FL121- | cagggatccgAATTCCatATGAGC | 69 | GT007 | 897 bp |
| | | futC-F | ATCATCCGTCT | | | |
| | | FL121- | GTGCGGCCGCAAGCttaGCAG | 70 | | |
| | | futC-R | CTGCTGTGTTTATCAAC | | | |
| | pET | FL121-F | ACAGCAGCTGCtaaGCTTGCG | 71 | pET28a | 5246 bp |
| | Back- | | GCCGCACTCGAGCAC | | | |
| | bone | FL121-R | tcttcgattttCATGGTATATCTCCT | 72 | | |
| | | | TCTTAAAGTTA | | | |
| pET- | SUMO1 | FL122- | AAGGAGATATACCatgtcggactc | 73 | SUMO1 | 294 bp |
| SUMO1- | | SUMO1-F | agaagtcaa | | | |
| futC | | FL122- | GATGCTCATatGaccaccaatctgttc | 74 | | |
| | | SUMO1-R | tctgt | | | |
| | futC | FL122- | acagattggtggtCatATGAGCATCA | 75 | GT007 | 897 bp |
| | | futC-F | TCCGTCT | | | |
| | | FL122- | GTGCGGCCGCAAGCttaGCAG | 76 | | |
| | | futC-R | CTGCTGTGTTTATCAAC | | | |
| | pET | FL122-F | ACAGCAGCTGCtaaGCTTGCG | 71 | pET28a | 5246 bp |
| | Back- | | GCCGCACTCGAGCAC | | | |
| | bone | FL122-R | ctgagtccgacatGGTATATCTCCT | 77 | | |
| | | | TCTTAAAGT | | | |
| pET- | SUMO2 | FL123- | gGAAGGAGATATACCATGG | 78 | SUMO2 | 306 bp |
| SUMO2- | | SUMO2-F | GCCATCATCATCACCA | | | |
| futC | | FL123- | TGATGCTCATatGACCACCGG | 79 | | |
| | | SUMO2-R | TCTGTTGCTGA | | | |
| | futC | FL123- | ACAGACCGGTGGTCatATGA | 80 | GT007 | 897 bp |
| | | futC-F | GCATCATCCGTCTG | | | |
| | | FL123- | GTGCGGCCGCAAGCttaGCAG | 81 | | |
| | | futC-R | CTGCTGTGTTTATCAAC | | | |
| | pET | FL123-F | ACAGCAGCTGCtaaGCTTGCG | 71 | pET28a | 5246 bp |
| | Back- | | GCCGCACTCGAGCAC | | | |
| | bone | FL123-R | ATGATGATGGCCCATGGTAT | 82 | | |
| | | | ATCTCCTTCTTAAAGTTA | | | |
| pET- | TrxA | FL124- | AGGAGATATACCatgagcgataaa | 83 | TrxA | 567 bp |
| TrxA- | | TrxA-F | attattca | | | |
| futC | | FL124- | TGATGCTCATatGgaatteggatccc | 84 | | |
| | | TrxA-R | tgaaaat | | | |
| | futC | FL124- | agggatccgaattcCatATGAGCATC | 85 | GT007 | 897 bp |
| | | futC-F | ATCCGTCT | | | |
| | | FL124- | GTGCGGCCGCAAGCttaGCAG | 86 | | |
| | | futC-R | CTGCTGTGTTTATCAAC | | | |
| | pET | FL124-F | ACAGCAGCTGCtaaGCTTGCG | 71 | pET28a | 5246 bp |
| | Back- | | GCCGCACTCGAGCAC | | | |
| | bone | FL124-R | ttttatcgctcatGGTATATCTCCTT | 87 | | |
| | | | CTTAAAG | | | |
| pET- | futC | FAB- | CGCGCGGCAGCCATCatATG | 88 | GT007 | 897 bp |
| futC | | futC-F | AGCATCATCCGTCTGCA | | | |
| | | 28A- | GTGCGGCCGCAAGCttaGCAG | 89 | | |
| | | GT008-R | CTGCTGTGTTTATCAAC | | | |
| | pET | 28A- | ACAGCAGCTGCtaaGCTTGCG | 71 | pET28a | 5246 bp |
| | Back- | GT008- | GCCGCACTCGAGCAC | | | |
| | bone | F2 | | | | |
| | | FAB-R1 | GATGATGCTCATatGATGGCT | 90 | | |
| | | | GCCGCGCGGCAC | | | |

### 2.2 Production of 2′-FL During Fermentation

2.2.1 Construction of 2′-FL Producing E. coli Strains

Competent cells were prepared based on the gene knockout strain FLIS009, the specific method was the same as that in 1.2.1, and then the plasmids pRSF-CBDG+pET-MBP-futC, pRSF-CBDG+pET-SUMO1-futC, pRSF-CBDG+pET-SUMO2-futC, pRSF-CBDG+pET-TrxA-futC, pRSF-CBDG+pET-futC were respectively transferred into FLIS009 competent cells, and screened for correct clones on LB plate (100 μg/ml ampicillin, 50 μg/ml kana antibiotics). The strain E. coli FLIS009-FL carrying the 2′-FL synthesis pathway was verified by PCR and named as FLIS201, FLIS202, FLIS203, FLIS204, FLIS205, respectively.

2.2.2 Producing 2′-FL with FLIS009-FL Strain
(1) TB medium: trypton 12 g (Trypton Oxoid LP0042 73049-73-7 BR), yeast extract 24 g, glycerol 4 ml, 2.31 g KH₂PO₄ and 12.54 g K₂HPO₄ were diluted to 1000 ml with deionized water, sterilized at 121° C. for 30 min, and stored at room temperature.
(2) LB medium: 10 g of tryptone was weighed respectively, distilled water was added at a ratio of 1:4 (mass to volume ratio, g/mL) to dissolve and mix, the pH was adjusted to 7.2 with 1 mol/L NaOH, and the liquid was diluted to 1 L, sterilized at 121° C. for 30 min, and stored at 4° C. without adding agar to the LB liquid.
(3) 1000 g/L glycerol: 1000 g glycerol was weighed, diluted to 1 L with deionized water, sterilized at 121° C. for 30 min, and stored at room temperature.
(4) 250 g/L lactose: 250 g lactose was dissolved in deionized water (dissolve by heating), diluted to 1 L, sterilized at 121° C. for 30 minutes, and stored at room temperature.
(5) Preparation of seed solution: the strains were inoculated into 5 mL of LB medium (containing 100 μg/ml ampicillin and 50 μg/ml kana antibiotics), and cultured at 37° C., 250 rpm for 4 hours.
(6) Fermentation culture: the seed liquid was inoculated into fresh fermentation medium (TB medium) with a ratio of seed liquid:medium=1:100 (v/v), cultivated at 37° C., 220 rpm until OD600 is 0.8, then IPTG (to a final concentration of 0.2 mM), 2 ml of 1000 g/L glycerol (to a final concentration of 20 g/L) and 4 ml of 250 g/L lactose (to a final concentration of 10 g/L) were added, the resultant was cultured at 25° C., 220 rpm to induce protein expression and fermentation culture.
(7) Sample processing method: 2-3 ml of fermentation broth was taken to lyse the cells by repeatedly freezing and thawing, the resultant was put in boiling water for 20 minutes after lysis, and then centrifuged (4° C., 12000 rpm for 5 minutes), the pellet was removed and the supernatant was kept and passed through a 0.22 μm filter membrane, and the content of 2′-FL in each treatment was detected by differential detection method.

### 2.3 Shake Flask Fermentation Validation

The strain obtained in 2.2.2 (1) was inoculated into TB medium according to 2.2.2(5), and cultured under the conditions of 25° C. and 220 rpm to induce protein expression and fermentation.
(2) The fermentation broth was taken for sample processing and 2′-FL content detection according to the method in 2.2.2 (6). The results are shown in Table 7.

TABLE 7
2′-FL yield

| | | 2′-FL |
|---|---|---|
| Strain | Plasmid | Yield (g/L) |
| FLIS201 | RSF-CBDG + pET-MBP-futC | 4.79 |
| FLIS202 | RSF-CBDG + pET-SUMO1-futC | 4.92 |
| FLIS203 | RSF-CBDG + pET-SUMO2-futC | 4.28 |
| FLIS204 | RSF-CBDG + pET-TrxA-futC | 4.09 |
| FLIS205 | RSF-CBDG + pET-futC | 2.25 |

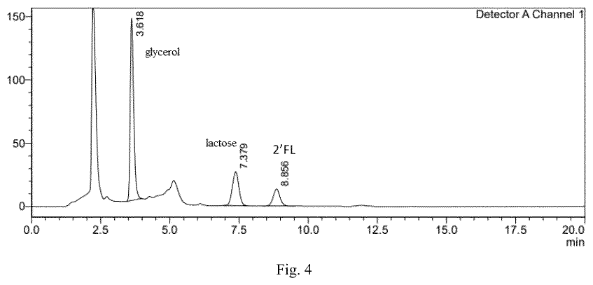

(3) From Table 7, it can be seen that the 2′-FL yield of tagged FLIS202 is significantly higher than that of untagged FLIS205, as shown in FIG. 4.

1. A genetically engineered bacterium, characterized in containing a gene encoding α-1,2-fucosyltransferase, and a gene encoding a protein tag is connected to the gene encoding α-1,2-fucosyltransferase, wherein the amino acid sequence of the α-1,2-fucosyl transferase is shown in SEQ ID NO: 1; the protein tag is MBP or TrxA, the amino acid sequence of the MBP is shown in SEQ ID NO: 2, and the amino acid sequence of the TrxA is shown in SEQ ID NO: 5.

2. The genetically engineered bacteria as claimed in claim 1, wherein the nucleotide sequence of the gene encoding the α-1,2-fucosyltransferase is shown in SEQ ID NO: 6;
and/or, the nucleotide sequence of the gene encoding the MBP is shown in SEQ ID NO: 7, and the nucleotide sequence of the gene encoding the TrxA is shown in SEQ ID NO: 10.

3. The genetically engineered bacteria as claimed in claim 1, wherein the GDP-fucose degradation pathway of the genetically engineered bacteria is blocked; preferably, all or part of the genes in the GDP-fucose degradation pathway of the genetically engineered bacteria are knocked out; more preferably, wcaJ gene of the genetically engineered bacteria is knocked out;
and/or, the GDP-mannose degradation pathway of the genetically engineered bacteria is blocked; preferably, all or part of the genes in the GDP-mannose degradation pathway of the genetically engineered bacteria are knocked out; more preferably, nudD and/or nudK genes of the genetically engineered bacteria are knocked out;
and/or, LacZ gene encoding the lactose operon s-galactosidase of the genetically engineered bacteria is knocked out;
and/or, the starting bacteria of the genetically engineered bacteria is Escherichia coli, preferably BL21 strain;
and/or, the genetically engineered bacteria overexpress one or more of manC, manB, gmd and wcaG genes, and the amino acid sequences encoded by the manC, manB, gmd and wcaG genes are respectively shown in SEQ ID NOs: 95-98; preferably, the nucleotide sequences of the manC, manB, gmd and wcaG genes are respectively shown in SEQ ID NOs: 91-94.

4. A preparation method of 2′-fucosyllactose, comprising: taking lactose as a substrate, glycerol or glucose as a carbon source, fermenting the genetically engineered bacteria as claimed in claim 1 to obtain the 2′-fucosyllactose; preferably, the fermentation medium is TB medium.

5. The preparation method as claimed in claim 4, wherein the genetically engineered bacteria are fermented until OD600 is 0.6-0.8, IPTG with a final concentration of 0.1-0.5 mM is added to the reaction system.

6. The preparation method as claimed in claim 5, wherein the concentration of the glycerol or glucose is 5-50 g/L, and the concentration of lactose is 5-20 g/L; and/or, when the IPTG is added, the temperature of the fermentation is adjusted to 20-30° C., and stirring is performed at a rotational speed of 150-300 rpm.

7. A recombinant expression vector comprising a gene encoding a protein tag and a gene encoding α-1,2-fucosyltransferase, the protein tag is MBP or TrxA, the amino acid sequence of the MBP is shown in SEQ ID NO: 2, and the amino acid sequence of the TrxA is shown in SEQ ID NO: 5, and the nucleotide sequence of the gene encoding the α-1,2-fucosyltransferase is shown in SEQ ID NO: 6;
preferably, the amino acid sequence of the α-1,2-fucosyltransferase is shown in SEQ ID NO: 1.

8. The recombinant expression vector as claimed in claim 7, wherein the nucleotide sequence of the gene encoding the MBP is shown in SEQ ID NO: 7, and the nucleotide sequence of the gene encoding the TrxA is shown in SEQ ID NO: 10;
preferably, the starting vector of the recombinant expression vector is pET28a plasmid vector.

9. A method for preparing the genetically engineered bacteria, comprising: transferring the recombinant expression vector as claimed in claim 7 into Escherichia coli to obtain the genetically engineered bacteria;
preferably, the method further comprises: knocking out the LacZ, wcaJ, nudD and/or nudK genes in the Escherichia coli; and/or, the method further comprises: overexpressing manC, manB, gmd and/or wcaG gene in the Escherichia coli, the amino acid sequences encoded by manC, manB, gmd and wcaG genes are respectively shown in SEQ ID NOs: 95-98.

10. Use of the genetically engineered bacteria as claimed in claim 1 in the preparation of fucosyllactose, the fucosyllactose is preferably 2′-fucosyllactose.

11. A preparation method of 2′-fucosyllactose, comprising: taking lactose as a substrate, glycerol or glucose as a carbon source, fermenting the genetically engineered bacteria as claimed in claim 2 to obtain the 2′-fucosyllactose; preferably, the fermentation medium is TB medium.

12. A preparation method of 2′-fucosyllactose, comprising: taking lactose as a substrate, glycerol or glucose as a carbon source, fermenting the genetically engineered bacteria as claimed in claim 3 to obtain the 2′-fucosyllactose; preferably, the fermentation medium is TB medium.

13. A method for preparing the genetically engineered bacteria, comprising: transferring the recombinant expression vector as claimed in claim 8 into Escherichia coli to obtain the genetically engineered bacteria;
preferably, the method further comprises: knocking out the LacZ, wcaJ, nudD and/or nudK genes in the Escherichia coli; and/or, the method further comprises: overexpressing manC, manB, gmd and/or wcaG gene in the Escherichia coli, the amino acid sequences encoded by manC, manB, gmd and wcaG genes are respectively shown in SEQ ID NOs: 95-98.

14. Use of the genetically engineered bacteria as claimed in claim 2 in the preparation of fucosyllactose, the fucosyllactose is preferably 2′-fucosyllactose.

15. Use of the genetically engineered bacteria as claimed in claim 3 in the preparation of fucosyllactose, the fucosyllactose is preferably 2′-fucosyllactose.

16. Use of the recombinant expression vector as claimed in claim 7 in the preparation of fucosyllactose, the fucosyllactose is preferably 2′-fucosyllactose.

17. Use of the recombinant expression vector as claimed in claim 8 in the preparation of fucosyllactose, the fucosyllactose is preferably 2′-fucosyllactose.


SEQUENCE LISTING (98 sequences)

SEQ ID NO: 1 - protein, 300 aa, synthetic construct
  REGION 1..300: note = alpha-1,2-fucosyltransferase futC (GT007) amino acid sequence
MAFKVVQICGGLGNQMFQYAFAKSLQKHLNTPVLLDTTSFDWSNRKMQLELFPIDLPYANAKEIAIAKMQHLPKLVRDALKYIGFDRVSQEIVFEYEPKLLKPSRLTYFFGYFQDPRYFDAISSLIKQTFTLPPPPENNKNNNKKEEEYQRKLSLILAAKNSVFVHIRRGDYVGIGCQLGIDYQKKALEYMAKRVPNMELFVFCEDLKFTQNLDLGYPFTDMTTRDKEEEAYWDMLLMQSCKHGIIANSTYSWWAAYLMENPEKIIIGPKHWLFGHENILCKEWVKIESHFEVKSQKYNA

SEQ ID NO: 2 - protein, 401 aa, synthetic construct
  REGION 1..401: note = MBP
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSGTGGGSGDDDDKSPMGEDIPTTENLYFQGSEF

SEQ ID NO: 3 - protein, 98 aa, synthetic construct
  REGION 1..98: note = SUMO1
MSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 4 - protein, 102 aa, synthetic construct
  REGION 1..102: note = SUMO2
MGHHHHHHGSLQEEKPKEGVKTENDHINLKVAGQDGSVVQFKIKRHTPLSKLMKAYCERQGLSMRQIRFRFDGQPINETDTPAQLEMEDEDTIDVFQQQTGG

SEQ ID NO: 5 - protein, 189 aa, synthetic construct
  REGION 1..189: note = TrxA
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTDDDDKAMGSHHHHHHHHGSDYDIPTTENLYFQGSEF

SEQ ID NO: 6 - DNA, 907 bp, synthetic construct
  misc_feature 1..907: note = alpha-1,2-fucosyltransferase futC (GT007) nucleotide sequence
  source 1..907: mol_type = other DNA
atggcgttcaaagttgttcagatctgcggcggtctgggtaaccagatgttccagtacgcgttcgcgaaaagcctgcagaaacacctgaacaccccggttctgctggataccaccagcttcgattggagcaaccgtaaaatgcagctggaactgttcccgatcgatctgccgtacgcgaacgcgaaagaaatcgcgatcgcgaaaatgcagcacctgccgaaactggttcgtgatgcgctgaaatacatcggcttcgatcgtgttagccaggaaatcgttttcgaatacgaaccgaaactgctgaaaccgagccgtctgacctacttcttcggttacttccaggacccgcgttacttcgatgcgatctctagcctgatcaaacagaccttcaccctgccgccgccgccggaaaacaacaaaaacaacaacaaaaaagaagaagaataccagcgtaaactgagcctgatcctggcggcgaaaaacagcgttttcgttcacatccgtcgtggtgactacgttggtatcggctgccagctgggtatcgattaccagaaaaaagcgctggaatacatggcgaaacgtgttccgaacatggaactgttcgttttctgcgaagatctgaaattcacccagaacctggatctgggctacccgttcaccgatatgaccacccgtgataaagaagaagaagcgtactgggatatgctgctgatgcagtcttgcaaacacggcatcatcgcgaacagcacctactcttggtgggcggcgtacctgatggaaaacccggaaaaaatcatcatcggtccgaaacactggctgttcggtcacgaaaacatcctgtgcaaagaatgggttaaaatcgaatcccacttcgaagttaaatctcagaaatacaacgcgtaagctt

SEQ ID NO: 7 - DNA, 1203 bp, synthetic construct
  misc_feature 1..1203: note = MBP
  source 1..1203: mol_type = other DNA
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgggtaccggtggtggctccggtgatgacgacgacaagagtcccatgggtgaagatatcccaacgaccgaaaacctgtattttcagggatccgaattc

SEQ ID NO: 8 - DNA, 294 bp, synthetic construct
  misc_feature 1..294: note = SUMO1
  source 1..294: mol_type = other DNA
atgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggt

SEQ ID NO: 9 - DNA, 306 bp, synthetic construct
  misc_feature 1..306: note = SUMO2
  source 1..306: mol_type = other DNA
atgggccatcatcatcaccatcacggcagcctgcaagaagagaaaccgaaagagggcgttaagaccgagaatgaccacattaacctgaaggtcgctggtcaagatggcagcgtggtgcagtttaagatcaagcgtcacacgccgttgagcaagctgatgaaggcttactgcgagcgtcagggtctgagcatgcgtcagatccgctttcgtttcgatggccagccgatcaatgagactgacaccccagcgcaactggagatggaagatgaagataccatcgacgtctttcagcaacagaccggtggt

SEQ ID NO: 10 - DNA, 567 bp, synthetic construct
  misc_feature 1..567: note = TrxA
  source 1..567: mol_type = other DNA
atgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggccatatgcaccatcatcatcatcattcttctggtctggtgccacgcggttctggtatgaaagaaaccgctgctgctaaattcgaacgccagcacatggacagcccagatctgggtaccgacgacgacgacaaggccatgggttcccaccatcaccatcaccatcaccacggttctgattacgatatcccaacgaccgaaaacctgtattttcagggatccgaattc

SEQ ID NO: 11 - DNA, 35 bp, synthetic construct
  misc_feature 1..35: note = primer GA001-P1-F4
  source 1..35: mol_type = other DNA
tgccgaccgtctagagtcgacctgcagaagcttag

SEQ ID NO: 12 - DNA, 45 bp, synthetic construct
  misc_feature 1..45: note = primer GA001-P1-R4
  source 1..45: mol_type = other DNA
aactggcgttacccaacttaatcactagtattatacctaggactg

SEQ ID NO: 13 - DNA, 47 bp, synthetic construct
  misc_feature 1..47: note = primer GA001-P1-F1
  source 1..47: mol_type = other DNA
tagtgattaagttgggtaacgccagttttagagctagaaatagcaag

SEQ ID NO: 14 - DNA, 35 bp, synthetic construct
  misc_feature 1..35: note = primer GA001-P1-R1
  source 1..35: mol_type = other DNA
gttccggaattcaaaaaaagcaccgactcggtgcc

SEQ ID NO: 15 - DNA, 39 bp, synthetic construct
  misc_feature 1..39: note = primer GA001-LF
  source 1..39: mol_type = other DNA
gctttttttgaattccggaacgggaaggcgactggagtg

SEQ ID NO: 16 - DNA, 37 bp, synthetic construct
  misc_feature 1..37: note = primer GA001-P1-LR
  source 1..37: mol_type = other DNA
ggtgcgggcctcgacggccagtgaatccgtaatcatg

SEQ ID NO: 17 - DNA, 35 bp, synthetic construct
  misc_feature 1..35: note = primer GA001-P1-RR
  source 1..35: mol_type = other DNA
tcgactctagacggtcggcaaagaccagaccgttc

SEQ ID NO: 18 - DNA, 32 bp, synthetic construct
  misc_feature 1..32: note = primer GA001-P1-RF
  source 1..32: mol_type = other DNA
ctggccgtcgaggcccgcaccgatcgcccttc

SEQ ID NO: 19 - DNA, 34 bp, synthetic construct
  misc_feature 1..34: note = primer nudK-f2
  source 1..34: mol_type = other DNA
tgaattcttcccttcctgaatcatctgcaaaaac

SEQ ID NO: 20 - DNA, 34 bp, synthetic construct
  misc_feature 1..34: note = primer nudK-R2
  source 1..34: mol_type = other DNA
gtggagtcggtaaaataacaataatatttcgttg

SEQ ID NO: 21 - DNA, 36 bp, synthetic construct
  misc_feature 1..36: note = primer nudK-F3
  source 1..36: mol_type = other DNA
attgttattttaccgactccacagcgcgaaatgaac

SEQ ID NO: 22 - DNA, 29 bp, synthetic construct
  misc_feature 1..29: note = primer nudK-R3
  source 1..29: mol_type = other DNA
ctagaccggaagagccgtttatcaatacc

SEQ ID NO: 23 - DNA, 39 bp, synthetic construct
  misc_feature 1..39: note = primer nudK-F4
  source 1..39: mol_type = other DNA
gataaacggctcttccggtctagagtcgacctgcagaag

SEQ ID NO: 24 - DNA, 51 bp, synthetic construct
  misc_feature 1..51: note = primer nudK-R4
  source 1..51: mol_type = other DNA
ctaaaacgcgcagctttcaatcagctgactagtattatacctaggactgag

SEQ ID NO: 25 - DNA, 52 bp, synthetic construct
  misc_feature 1..52: note = primer nudK-F1
  source 1..52: mol_type = other DNA
ctagtcagctgattgaaagctgcgcgttttagagctagaaatagcaagttaa

SEQ ID NO: 26 - DNA, 46 bp, synthetic construct
  misc_feature 1..46: note = primer nudK-R1
  source 1..46: mol_type = other DNA
gattcaggaagggaagaattcaaaaaaagcaccgactcggtgccac

SEQ ID NO: 27 - DNA, 39 bp, synthetic construct
  misc_feature 1..39: note = primer pT-nudD-F2
  source 1..39: mol_type = other DNA
gctttttttgaattccacttcgtaatcctgaatatgcag

SEQ ID NO: 28 - DNA, 37 bp, synthetic construct
  misc_feature 1..37: note = primer pT-nudD-R2
  source 1..37: mol_type = other DNA
cgctccactgattaccactggctgacgccggacgcac

SEQ ID NO: 29 - DNA, 43 bp, synthetic construct
  misc_feature 1..43: note = primer pT-nudD-F3
  source 1..43: mol_type = other DNA
ccagtggtaatcagtggagcgcactaccgtggcaaagtcttcc

SEQ ID NO: 30 - DNA, 40 bp, synthetic construct
  misc_feature 1..40: note = primer pT-nudD-R3
  source 1..40: mol_type = other DNA
cgactctagagacaacttccacccgagtaattcgcatgtg

SEQ ID NO: 31 - DNA, 48 bp, synthetic construct
  misc_feature 1..48: note = primer pT-nudD-F1
  source 1..48: mol_type = other DNA
ctagtgtgagtggtgaaatccgtgcgttttagagctagaaatagcaag

SEQ ID NO: 32 - DNA, 37 bp, synthetic construct
  misc_feature 1..37: note = primer pT-nudD-R1
  source 1..37: mol_type = other DNA
gattacgaagtggaattcaaaaaaagcaccgactcgg

SEQ ID NO: 33 - DNA, 39 bp, synthetic construct
  misc_feature 1..39: note = primer pT-nudD-F4
  source 1..39: mol_type = other DNA
ggtggaagttgtctctagagtcgacctgcagaagcttag

SEQ ID NO: 34 - DNA, 52 bp, synthetic construct
  misc_feature 1..52: note = primer pT-nudD-R4
  source 1..52: mol_type = other DNA
ctaaaacgcacggatttcaccactcacactagtattatacctaggactgagc

SEQ ID NO: 35 - DNA, 48 bp, synthetic construct
  misc_feature 1..48: note = primer WcaJ-F2
  source 1..48: mol_type = other DNA
cgagtcggtgctttttttgaattcgacagcggcatgatcccgtggctg

SEQ ID NO: 36 - DNA, 36 bp, synthetic construct
  misc_feature 1..36: note = primer wacj-R2
  source 1..36: mol_type = other DNA
cgccacgccagcccaacaggtgcatgtagaggaatg

SEQ ID NO: 37 - DNA, 37 bp, synthetic construct
  misc_feature 1..37: note = primer wcaJ-F3
  source 1..37: mol_type = other DNA
catgcacctgttgggctggcgtggcgaaaccgacacg

SEQ ID NO: 38 - DNA, 44 bp, synthetic construct
  misc_feature 1..44: note = primer wcaJ-R3
  source 1..44: mol_type = other DNA
cagggtaatagatctaagcttgcgcggaactgctgtccgtgggg

SEQ ID NO: 39 - DNA, 51 bp, synthetic construct
  misc_feature 1..51: note = primer wcaJ-F1
  source 1..51: mol_type = other DNA
gtcctaggtataatactagtcatcgccgcagcggtttcaggttttagagct

SEQ ID NO: 40 - DNA, 48 bp, synthetic construct
  misc_feature 1..48: note = primer wcaJ-R1
  source 1..48: mol_type = other DNA
cagccacgggatcatgccgctgtcgaattcaaaaaaagcaccgactcg

SEQ ID NO: 41 - DNA, 44 bp, synthetic construct
  misc_feature 1..44: note = primer wcaJ-F4
  source 1..44: mol_type = other DNA
ccccacggacagcagttccgcgcaagcttagatctattaccctg

SEQ ID NO: 42 - DNA, 45 bp, synthetic construct
  misc_feature 1..45: note = primer wcaJ-R4
  source 1..45: mol_type = other DNA
gctctaaaacctgaaaccgctgcggcgatgactagtattatacct

SEQ ID NO: 43 - DNA, 29 bp, synthetic construct
  misc_feature 1..29: note = primer LacZ-YZ1-F
  source 1..29: mol_type = other DNA
cgcgctgttagcgggcccattaagttctg

SEQ ID NO: 44 - DNA, 28 bp, synthetic construct
  misc_feature 1..28: note = primer LAC-YZ2-R
  source 1..28: mol_type = other DNA
ggtcttcatccacgcgcgcgtacatcgg

SEQ ID NO: 45 - DNA, 24 bp, synthetic construct
  misc_feature 1..24: note = primer wcaJ-YZ-for
  source 1..24: mol_type = other DNA
gtcggcctgttggcagaagcattc

SEQ ID NO: 46 - DNA, 29 bp, synthetic construct
  misc_feature 1..29: note = primer wcaJ-YZ-R
  source 1..29: mol_type = other DNA
gtagccaaacagcagcgttcttaccgcac

SEQ ID NO: 47 - DNA, 23 bp, synthetic construct
  misc_feature 1..23: note = primer nudD-YZ-F
  source 1..23: mol_type = other DNA
ccgtcgccagctgtgccactttg

SEQ ID NO: 48 - DNA, 26 bp, synthetic construct
  misc_feature 1..26: note = primer nudD-YZ-R
  source 1..26: mol_type = other DNA
caaactgtgcgaatcttacaatcgcc

SEQ ID NO: 49 - DNA, 27 bp, synthetic construct
  misc_feature 1..27: note = primer nudK-YZ-F
  source 1..27: mol_type = other DNA
gctgagcatcaataaacaacaacgctg

SEQ ID NO: 50 - DNA, 24 bp, synthetic construct
  misc_feature 1..24: note = primer nudK-YZ-R
  source 1..24: mol_type = other DNA
atgaagatgcgccgggcgtttatg

SEQ ID NO: 51 - DNA, 19 bp, synthetic construct
  misc_feature 1..19: note = primer CX-targetF-F
  source 1..19: mol_type = other DNA
cagcgagtcagtgagcgag

SEQ ID NO: 52 - DNA, 19 bp, synthetic construct
  misc_feature 1..19: note = primer CX-targetF-R
  source 1..19: mol_type = other DNA
gacattgcactccaccgct

SEQ ID NO: 53 - DNA, 20 bp, synthetic construct
  misc_feature 1..20: note = primer Kan-F
  source 1..20: mol_type = other DNA
gaaggagaaaactcaccgag

SEQ ID NO: 54 - DNA, 33 bp, synthetic construct
  misc_feature 1..33: note = primer Pcr4-R1
  source 1..33: mol_type = other DNA
cagctgcataaaattgcgattggcaaaaccatc

SEQ ID NO: 55 - DNA, 34 bp, synthetic construct
  misc_feature 1..34: note = primer CBGW-manC-F
  source 1..34: mol_type = other DNA
taaggagatataccatggcgcagtcgaaactcta

SEQ ID NO: 56 - DNA, 45 bp, synthetic construct
  misc_feature 1..45: note = primer CBGW-manC-R
  source 1..45: mol_type = other DNA
gttaattttttcatggtatatctccttttacacccgtccgtagcg

SEQ ID NO: 57 - DNA, 51 bp, synthetic construct
  misc_feature 1..51: note = primer CBGW-manB-F
  source 1..51: mol_type = other DNA
cggacgggtgtaaaaggagatataccatgaaaaaattaacctgctttaaag

SEQ ID NO: 58 - DNA, 33 bp, synthetic construct
  misc_feature 1..33: note = primer CBGW-manB-R
  source 1..33: mol_type = other DNA
gagctcgaattcttactcgttcagcaacgtcag

SEQ ID NO: 59 - DNA, 34 bp, synthetic construct
  misc_feature 1..34: note = primer CBGW-gmd-F
  source 1..34: mol_type = other DNA
gaaggagatatacaatgtcaaaagtcgctctcat

SEQ ID NO: 60 - DNA, 46 bp, synthetic construct
  misc_feature 1..46: note = primer CBGW-gmd-R
  source 1..46: mol_type = other DNA
gttgtttactcatggtatatctccttttatgactccagcgcgatcg

SEQ ID NO: 61 - DNA, 50 bp, synthetic construct
  misc_feature 1..50: note = primer CBGW-wcaG-F
  source 1..50: mol_type = other DNA
ctggagtcataaaaggagatataccatgagtaaacaacgagtttttattg

SEQ ID NO: 62 - DNA, 34 bp, synthetic construct
  misc_feature 1..34: note = primer CBGW-wcaG-R
  source 1..34: mol_type = other DNA
tggcagcagcctaggttacccccgaaagcggtct

SEQ ID NO: 63 - DNA, 32 bp, synthetic construct
  misc_feature 1..32: note = primer CBGW-F1
  source 1..32: mol_type = other DNA
gctttcgggggtaacctaggctgctgccaccg

SEQ ID NO: 64 - DNA, 38 bp, synthetic construct
  misc_feature 1..38: note = primer CBGW-R1
  source 1..38: mol_type = other DNA
cgactgcgccatggtatatctccttattaaagttaaac

SEQ ID NO: 65 - DNA, 33 bp, synthetic construct
  misc_feature 1..33: note = primer CBGW-F2
  source 1..33: mol_type = other DNA
ttgctgaacgagtaagaattcgagctcggcgcg

SEQ ID NO: 66 - DNA, 39 bp, synthetic construct
  misc_feature 1..39: note = primer CBGW-R2
  source 1..39: mol_type = other DNA
gcgacttttgacattgtatatctccttcttatacttaac

SEQ ID NO: 67 - DNA, 33 bp, synthetic construct
  misc_feature 1..33: note = primer FL121-MBP-F
  source 1..33: mol_type = other DNA
aaggagatataccatgaaaatcgaagaaggtaa

SEQ ID NO: 68 - DNA, 32 bp, synthetic construct
  misc_feature 1..32: note = primer FL121-MBP-R
  source 1..32: mol_type = other DNA
gatgctcatatggaattcggatccctgaaaat

SEQ ID NO: 69 - DNA, 35 bp, synthetic construct
  misc_feature 1..35: note = primer FL121-futC-F
  source 1..35: mol_type = other DNA
cagggatccgaattccatatgagcatcatccgtct

SEQ ID NO: 70 - DNA, 38 bp, synthetic construct
  misc_feature 1..38: note = primer FL121-futC-R
  source 1..38: mol_type = other DNA
gtgcggccgcaagcttagcagctgctgtgtttatcaac

SEQ ID NO: 71 - DNA, 36 bp, synthetic construct
  misc_feature 1..36: note = primer FL121-F
  source 1..36: mol_type = other DNA
acagcagctgctaagcttgcggccgcactcgagcac

SEQ ID NO: 72 - DNA, 37 bp, synthetic construct
  misc_feature 1..37: note = primer FL121-R
  source 1..37: mol_type = other DNA
tcttcgattttcatggtatatctccttcttaaagtta

SEQ ID NO: 73 - DNA, 33 bp, synthetic construct
  misc_feature 1..33: note = primer FL122-SUMO1-F
  source 1..33: mol_type = other DNA
aaggagatataccatgtcggactcagaagtcaa

SEQ ID NO: 74 - DNA, 32 bp, synthetic construct
  misc_feature 1..32: note = primer FL122-SUMO1-R
  source 1..32: mol_type = other DNA
gatgctcatatgaccaccaatctgttctctgt

SEQ ID NO: 75 - DNA, 33 bp, synthetic construct
  misc_feature 1..33: note = primer FL122-futC-F
  source 1..33: mol_type = other DNA
acagattggtggtcatatgagcatcatccgtct

SEQ ID NO: 76 - DNA, 38 bp, synthetic construct; identical to SEQ ID NO: 70
  misc_feature 1..38: note = primer FL122-futC-R
  source 1..38: mol_type = other DNA

SEQ ID NO: 77 - DNA, 34 bp, synthetic construct
  misc_feature 1..34: note = primer FL122-R
  source 1..34: mol_type = other DNA
ctgagtccgacatggtatatctccttcttaaagt

SEQ ID NO: 78 - DNA, 35 bp, synthetic construct
  misc_feature 1..35: note = primer FL123-SUMO2-F
  source 1..35: mol_type = other DNA
ggaaggagatataccatgggccatcatcatcacca

SEQ ID NO: 79 - DNA, 32 bp, synthetic construct
  misc_feature 1..32: note = primer FL123-SUMO2-R
  source 1..32: mol_type = other DNA
tgatgctcatatgaccaccggtctgttgctga

SEQ ID NO: 80 - DNA, 34 bp, synthetic construct
  misc_feature 1..34: note = primer FL123-futC-F
  source 1..34: mol_type = other DNA
acagaccggtggtcatatgagcatcatccgtctg

SEQ ID NO: 81 - DNA, 38 bp, synthetic construct; identical to SEQ ID NO: 70
  misc_feature 1..38: note = primer FL123-futC-R
  source 1..38: mol_type = other DNA

SEQ ID NO: 82 - DNA, 38 bp, synthetic construct
  misc_feature 1..38: note = primer FL123-R
  source 1..38: mol_type = other DNA
atgatgatggcccatggtatatctccttcttaaagtta

SEQ ID NO: 83 - DNA, 32 bp, synthetic construct
  misc_feature 1..32: note = primer FL124-TrxA-F
  source 1..32: mol_type = other DNA
aggagatataccatgagcgataaaattattca

SEQ ID NO: 84 - DNA, 33 bp, synthetic construct
  misc_feature 1..33: note = primer FL124-TrxA-R
  source 1..33: mol_type = other DNA
tgatgctcatatggaattcggatccctgaaaat

SEQ ID NO: 85 - DNA, 34 bp, synthetic construct
  misc_feature 1..34: note = primer FL124-futC-F
  source 1..34: mol_type = other DNA
agggatccgaattccatatgagcatcatccgtct

SEQ ID NO: 86 - DNA, 38 bp, synthetic construct; identical to SEQ ID NO: 70
  misc_feature 1..38: note = primer FL124-futC-R
  source 1..38: mol_type = other DNA

SEQ ID NO: 87 - DNA, 33 bp, synthetic construct
  misc_feature 1..33: note = primer FL124-R
  source 1..33: mol_type = other DNA
ttttatcgctcatggtatatctccttcttaaag

SEQ ID NO: 88 - DNA, 37 bp, synthetic construct
  misc_feature 1..37: note = primer FAB-futC-F
  source 1..37: mol_type = other DNA
cgcgcggcagccatcatatgagcatcatccgtctgca

SEQ ID NO: 89 - DNA, 38 bp, synthetic construct; identical to SEQ ID NO: 70
  misc_feature 1..38: note = primer 28A-GT008-R
  source 1..38: mol_type = other DNA

SEQ ID NO: 90 - DNA, 33 bp, synthetic construct
  misc_feature 1..33: note = primer FAB-R1
  source 1..33: mol_type = other DNA
gatgatgctcatatgatggctgccgcgcggcac

SEQ ID NO: 91 - DNA, 1437 bp, synthetic construct
  misc_feature 1..1437: note = manC gene
  source 1..1437: mol_type = other DNA
atggcgcagtcgaaactctatccagttgtgatggcaggtggctccggtagccgcttatggccgctttcccgcgtactttaccccaagcagtttttatgcctgaaaggcgatctcaccatgctgcaaaccaccatctgccgcctgaacggtgtggagtgcgaaagcccggtggtgatttgcaatgagcagcaccgctttattgtcgcggaacagctgcgtcaactgaacaaactcaccaagaacattattctcgaaccggcagggcgtaacactgcacctgccattgcgctggcggcgctggcggcaaaacgtcatagcccggagagcgacccgttaatgctggtcttggcggcggatcatgtgattgccgatgaagacgcgttccgtgccgccgtgcgtaatgccatgccgtatgccaaagcgggcaagctggtgaccttcggcattgtgccggatctacctgaaaccggttatggctatattcgtcgcggtgaagtgtcggcgggtgagcaggatacggtggcctttgaagtggcgcagtttgtcgaaaaaccgaatctggaaaccgctcaggcctatgtggcaagcggcgaatattactggaacagcggtatgttcctgttccgcgccggacgctatctcgaagaactgaaaaaatatcgcccggatattctcgatgcctgtgaaaaagcgatgagcgccgtcgatccggatctcgattttattcgtgtggatgaagaagcgtttctcgcctgcccggaagagtcggtggattacgcggtcatggaacgtacggcagatgccgttgtggtgccgatggatgcgggctggagtgatgtcggttcttggtcttcattatgggagatcagcgcccacaccgccgagggcaacgtttgccacggcgatgtgattaatcacaaaactgaaaacagctatgtgtacgccgaatctggcctggtcaccaccgtcggggtgaaagatttggtggtagtgcagaccaaagatgcagtgctgattgccgaccgtaacgcggtgcaggatgtgaaaaaagtggtcgagcagatcaaagccgatggtcgccatgagcatcgggtacatcgcgaagtgtatcgtccgtggggcaaatatgactctatcgacgcgggcgaccgctaccaggtgaaacgcatcaccgtgaaaccgggcgagggcttgtcggtacagatgcaccatcaccgcgcggaacactgggtagtggtcgcgggaacggcaaaagtcactattgacggtgatatcaaactgcttggtgaaaacgagtccatttatattccgctgggggcgacgcactgcctggaaaacccggggaaaattccgctcgatttaattgaagtgcggtccggctcttatctcgaagaggatgatgtggtgcgcttcgcggatcgctacggacgggtgtaa

SEQ ID NO: 92 - DNA, 1371 bp, synthetic construct
  misc_feature 1..1371: note = manB gene
  source 1..1371: mol_type = other DNA
atgaaaaaattaacctgctttaaagcctatgatattcgtggaaaattaggcgaagaactgaatgaagatattgcctggcgcattggtcgcgcttatggcgaatttctcaaaccgaaaaccattgtgttaggcggtgatgtccgcctcaccagcgaaaccttaaaactggcgctggcgaaaggtttacaggatgcgggcgtcgatgtgctggatattggcatgtccggcaccgaagagatctatttcgccacgttccatctcggtgtggatggcggcattgaagttaccgccagccataatccgatggattataacggcatgaagctggtgcgcgaaggggctcgcccgatcagcggtgataccggactgcgcgatgtccagcgtctggcagaagccaacgactttcctcccgttgatgaaaccaaacgcggtcgctatcagcaaatcaatctgcgtgacgcttacgttgatcacctgttcggttatatcaacgtcaaaaacctcacgccgctcaagctggtgatcaactccgggaacggcgcagcgggtccggtggtggacgctatcgaagcccgctttaacgccctcggcgctccggtggaattaatcaaagtgcacaacacgccggacggcaatttccccaacggtattcctaacccgctgctgccggaatgccgcgacgacacccgcaatgcggtcatcaaacacggcgcggatatgggcattgcctttgacggtgattttgatcgctgtttcctgtttgacgaaaaagggcagtttatcgagggctactacattgtcggcctgttggcagaagcattcctcgaaaaaaatcccggcgcgaagatcatccacgatccacgtctctcctggaacaccgttgatgtggtgactaccgcaggtggcaccccggtaatgtcgaaaaccggacacgcctttattaaagaacgtatgcgcaaggaagacgccatctacggtggcgaaatgagcgcccaccattacttccgtgatttcgcttactgcgacagcggcatgatcccgtggctgctggtcgccgaactggtgtgcctgaaagagaaaacgctgggcgaactggtacgcgaccggatggcggcgtttccggcaagcggtgagatcaacagcaaactggcgcaacccgttgaggcgattaaccgcgtcgaacagcattttagccgcgaggcgctggcggtggatcgcactgatggcatcagcatgacctttgccgactggcgctttaacctgcgcacctccaataccgaaccggtggtgcgcctgaatgtggaatcgcgcggtgatgtgccgctgatggaagcgcgaacgcgaactctgctgacgttgctgaacgagtaa

SEQ ID NO: 93 - DNA, 1122 bp, synthetic construct
  misc_feature 1..1122: note = gmd gene
  source 1..1122: mol_type = other DNA
atgtcaaaagtcgctctcatcaccggtgtaaccggacaagacggttcttacctggcagagtttctgctggaaaaaggttacgaggtgcatggtattaagcgtcgtgcatcgtcattcaacaccgagcgcgtggatcacatttatcaggatccgcacacctgcaacccgaaattccatctgcattatggcgacctgagtgatacctccaacctgacacgcattttgcgtgaagtgcagccggatgaagtgtataacctgggcgcaatgagccacgttgcggtctcttttgagtcaccggaatataccgcagacgttgatgcgatgggtacgctgcgcctgctcgaggcgatccgcttcctcggtctggaaaagaaaacccgtttttatcaggcttccacctctgaactgtacggtctggtgcaggaaattccgcagaaagaaactacgccgttctacccgcgatctccgtatgcggtcgccaaactgtacgcctactggatcaccgttaactaccgcgaatcctacggcatgtacgcctgtaacggtattctcttcaaccatgaatccccgcgccgcggtgaaaccttcgttacccgcaaaatcacccgcgcaatcgccaatatcgcccaggggctggagtcgtgcctgtacctcggcaatatggattccctgcgtgactggggccatgccaaagactacgtaaaaatgcagtggatgatgctgcaacaggaacagccggaagatttcgttattgctaccggcgttcagtactccgtacgtcagttcgtggaaatggcggcagcacagttgggcatcaaactgcgctttgaaggcacgggtgttgaagagaagggcattgtggtttccgtcaccgggcatgacgcgccgggcgttaaaccgggtgatgtgattatcgccgttgacccgcgttacttccgtccggcagaagttgaaacgctgctcggcgacccgaccaaagcgcacgaaaaactgggctggaaaccggaaatcaccctcagagagatggtgtctgaaatggtggctaatgacctcgaagcggcgaaaaaacactctctgctgaaatctcacggctacgacgtggcgatcgcgctggagtcataa

SEQ ID NO: 94 - DNA, 965 bp, synthetic construct
  misc_feature 1..965: note = wcaG gene
  source 1..965: mol_type = other DNA
atgagtaaacaacgagtttttattgctggtcatcgcgggatggtcggttctgccatcaggcggcagctcgaacagcgcggtgatgtggaactggtattacgcacccgcgacgagctgaacctgttggacagccgcgcggtgcatgatttctttgccagcgaacgcattgaccaggtctatctggcggcggcgaaagtgggcggcattgttgctaacaacacctatccggcggatttcatctaccagaacatgatgattgagagcaacatcattcacgccgcgcatcagaacgacgtgaacaaactgctgtttctcggatcgtcctgtatctacccgaaactggcaaaacagccgatggcagaaagcgagttgttgcagggcacgctggagccgactaacgagccttatgctattgccaaaatcgccgggatcaaactgtgcgaatcttacaatcgccagtacggacgagattaccgttcagtcatgccgaccaacctgtacgggccgcacgacaacttccacccgagtaattcgcatgtgatcccagcattgctgcgccgcttccacgaggcgacggcacagaatgcaccggacgtggtggtatggggcagcggtacaccgatgcgtgaattcctgcacgtcgatgatatggcggcggcgagcattcatgtcatggagctggcgcatgaagtctggctggagaacacccagccgatgctgtcgcacattaacgtcggcacgggcgttgactgcaccatccgtgaactggcgcaaaccatcgccaaagtggtgggttacaaaggtcgggtggtttttgatgccagcaaaccggatggtacgccgcgcaaactgctggatgtgacgcgcctgcatcagcttggctggtatcacgaaatctcactggaagcggggcttgccagcacttaccagtggttccttgagaatcaagaccgctttcgggggta

SEQ ID NO: 95 - protein, 478 aa, synthetic construct
  REGION 1..478: note = manC protein
MAQSKLYPVVMAGGSGSRLWPLSRVLYPKQFLCLKGDLTMLQTTICRLNGVECESPVVICNEQHRFIVAEQLRQLNKLTKNIILEPAGRNTAPAIALAALAAKRHSPESDPLMLVLAADHVIADEDAFRAAVRNAMPYAKAGKLVTFGIVPDLPETGYGYIRRGEVSAGEQDTVAFEVAQFVEKPNLETAQAYVASGEYYWNSGMFLFRAGRYLEELKKYRPDILDACEKAMSAVDPDLDFIRVDEEAFLACPEESVDYAVMERTADAVVVPMDAGWSDVGSWSSLWEISAHTAEGNVCHGDVINHKTENSYVYAESGLVTTVGVKDLVVVQTKDAVLIADRNAVQDVKKVVEQIKADGRHEHRVHREVYRPWGKYDSIDAGDRYQVKRITVKPGEGLSVQMHHHRAEHWVVVAGTAKVTIDGDIKLLGENESIYIPLGATHCLENPGKIPLDLIEVRSGSYLEEDDVVRFADRYGRV

SEQ ID NO: 96 - protein, 456 aa, synthetic construct
  REGION 1..456: note = manB protein
MKKLTCFKAYDIRGKLGEELNEDIAWRIGRAYGEFLKPKTIVLGGDVRLTSETLKLALAKGLQDAGVDVLDIGMSGTEEIYFATFHLGVDGGIEVTASHNPMDYNGMKLVREGARPISGDTGLRDVQRLAEANDFPPVDETKRGRYQQINLRDAYVDHLFGYINVKNLTPLKLVINSGNGAAGPVVDAIEARFNALGAPVELIKVHNTPDGNFPNGIPNPLLPECRDDTRNAVIKHGADMGIAFDGDFDRCFLFDEKGQFIEGYYIVGLLAEAFLEKNPGAKIIHDPRLSWNTVDVVTTAGGTPVMSKTGHAFIKERMRKEDAIYGGEMSAHHYFRDFAYCDSGMIPWLLVAELVCLKEKTLGELVRDRMAAFPASGEINSKLAQPVEAINRVEQHFSREALAVDRTDGISMTFADWRFNLRTSNTEPVVRLNVESRGDVPLMEARTRTLLTLLNE

SEQ ID NO: 97 - protein, 373 aa, synthetic construct [sequence omitted]

SEQ ID NO: 98 - protein, 321 aa, synthetic construct
  REGION 1..321: note = wcaG protein
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG